(19) United States
(12) Patent Application Publication
(10) Pub. No.: US 2025/0188004 A1
(43) Pub. Date: Jun. 12, 2025

(54) INTEGRATED NAPHTHA REFORMING AND BENZYL TOLUENE PRODUCTION PROCESS

(71) Applicant: Saudi Arabian Oil Company, Dhahran (SA)

(72) Inventors: Guanghui Zhu, Dhahran (SA); Anas S. Al-Aqeeli, Dhahran (SA); Faisal M. Almulla, Dhahran (SA); Raed H. Abudawoud, Dhahran (SA)

(73) Assignee: Saudi Arabian Oil Company, Dhahran (SA)

(21) Appl. No.: 18/530,454

(22) Filed: Dec. 6, 2023

(51) Int. Cl. C07C 6/12; B01J 19/24; B01J 21/04; B01J 23/42; B01J 38/42; C07C 1/30; C07C 5/10; C07C 17/14; C10G 35/085

(52) U.S. Cl. CPC C07C 6/126; B01J 19/2445; B01J 19/245; B01J 21/04; B01J 23/42; B01J 38/42; C07C 1/30; C07C 5/10; C07C 17/14; C10G 35/085; B01J 2219/00038; B01J 2219/0004; C10G 2300/70

(57) ABSTRACT

An integrated process and associated system for naphtha reforming and benzyl toluene production. The process includes providing a first toluene stream and a chlorine gas stream to a halogenation reactor to generate a benzyl chloride stream and a first HCl effluent stream then providing the benzyl chloride stream, a second toluene stream, and a Lewis acid stream to an alkylation reactor to generate a benzyl toluene stream and a second HCl effluent stream through a Friedel-Crafts reaction. The process further includes providing naphtha to a catalytic reforming unit to generate a reformate stream and a spent catalyst stream and then providing the spent catalyst stream and at least one of the first HCl effluent stream and the second HCl effluent stream to a catalyst regenerator to regenerate the spent catalyst stream.

### TECHNICAL FIELD

The present disclosure relates to an integrated process and associated system for naphtha reforming and benzyl toluene production.

### BACKGROUND

Aromatics such as BTX (benzene, toluene, and xylenes) and olefins are valuable chemicals frequently utilized in the production of many materials and formulation of many consumer goods. For example, BTX compounds are frequently utilized during the processing or production of petroleum products and during the production of consumer goods such as paints and lacquers, thinners, fuels, rubber products, adhesives, inks, cosmetics and pharmaceutical products. Similarly, light olefins are the building blocks for many modern plastic products. One of the most common ways to generate BTX compounds is through reforming naphtha. As such, naphtha reforming processes are commonly included in hydrocarbon refineries.

Additionally, Benzyl Toluene is one of the most promising carriers for hydrogen transportation due to its high boiling point, low melting point, low flammability, high flash point and high hydrogen storage density. Therefore, it is expected the demand for Benzyl Toluene will steadily increase over time. As such, benzyl toluene generation processes are expected to develop an increased presence in hydrocarbon refineries.

It is desirable to be able to efficiently generate BTX and benzyl toluene in view of the current and expected increasing demand for such valuable chemicals. However, existing refining systems and processes do not maximize the economic synergy afforded by integration of the processes.

### SUMMARY

Accordingly, there is a clear and long-standing need to provide an efficient and economical process for the production of both BTX and benzyl toluene. The process and system of the present disclosure address this clear and long-standing need by utilizing processes and systems which integrate both production processes to provide mutually beneficial utilization of waste streams from one process as a feed to the other process. Specifically, regeneration of reforming catalysts utilized in the naphtha reforming process requires a steady supply of chlorine which can be costly to generate, store, and/or transport. However, the integrated processes of the present disclosure allow for direct transfer of waste HCl to a catalyst regenerator without the need for expensive acquisition and/or transport and without generation of excess carbon dioxide. Similarly, toluene generated as part of the naphtha reforming may be directly utilized in the benzyl toluene generation process correspondingly eliminating or reducing generation, storage, and/or transportation costs.

In accordance with one or more embodiments of the present disclosure, an integrated naphtha reforming and benzyl toluene production process is disclosed. The process includes (i) providing a first toluene stream and a chlorine gas stream to a halogenation reactor; (ii) operating the halogenation reactor to generate a benzyl chloride stream and a first HCl effluent stream; (iii) providing the benzyl chloride stream, a second toluene stream, and a Lewis acid stream to an alkylation reactor; (iv) operating the alkylation reactor to generate a benzyl toluene stream and a second HCl effluent stream through a Friedel-Crafts reaction; (v) providing naphtha to a catalytic reforming unit, wherein a reforming catalyst is disposed within the catalytic reforming unit; (vi) operating the catalytic reforming unit to generate a reformate stream and a spent catalyst stream, the spent catalyst stream representing the used reforming catalyst; (vii) providing the spent catalyst stream and at least one of the first HCl effluent stream and the second HCl effluent stream to a catalyst regenerator; (viii) operating the catalyst regenerator to regenerate the reforming catalyst and form a regenerated reforming catalyst stream; and (ix) providing the regenerated reforming catalyst stream as a recycle stream to the catalytic reforming unit.

In additional embodiments, the reformate stream is provided to an aromatics complex to separate and capture benzene, toluene, and mixed xylenes; and the toluene captured in the aromatics complex is provided to at least one of the halogenation reactor and the alkylation reactor at least partially in lieu of the first toluene stream and the second toluene stream.

In accordance with one or more embodiments of the present disclosure, an integrated system for naphtha reforming and benzyl toluene production is disclosed. The system includes (i) a catalytic reforming unit comprising an input to receive a naphtha feed stream, a reforming catalyst disposed within the catalytic reforming unit, and two or more outlets to discharge a spent catalyst stream and a reformate stream, wherein the catalytic reforming unit is configured to reform the naphtha feed stream to generate the reformate stream and the spent catalyst stream; (ii) a catalyst regenerator fluidly connected to the catalytic reforming unit to receive the spend catalyst stream, wherein the catalyst regenerator is configured to regenerate the spent catalyst; (iii) a halogenation reactor comprising an input for receiving a first toluene stream, an input for receiving a chlorine stream, an output for discharging a first HCl effluent stream, and an output for discharging a benzyl chloride stream; (iv) an alkylation reactor comprising an input to receive the benzyl chloride stream from the halogenation reactor, an input to receive a Lewis acid stream, an input to receive a second toluene stream, an output to discharge a benzyl toluenes stream, and an output to discharge a second HCl effluent stream, wherein the alkylation reactor operates based on a Friedel-Crafts reaction; and (v) one or more HCl recycling lines fluidly connecting the catalyst regenerator with the halogenation reactor and the alkylation reactor for transport of the first HCl effluent stream and the second HCl effluent stream to an input of the catalyst regenerator.

In additional embodiments, the system further comprises an aromatics complex configured to receive and separate the reformate steam into a benzene stream, a third toluene stream, and mixed xylenes stream; and a fluid connection between the halogenation reactor and the alkylation reactor for transfer of the third toluene stream from the aromatics complex to at least one of the halogenation reactor and the alkylation reactor at least partially in lieu of the first toluene stream and the second toluene stream.

Additional features and advantages of the described embodiments will be set forth in the detailed description that follows. The additional features and advantages of the described embodiments will be, in part, readily apparent to those skilled in the art from that description or recognized by practicing the described embodiments, including the detailed description that follows as well as the drawings and the claims.

### BRIEF DESCRIPTION OF THE DRAWINGS

The following detailed description of specific embodiments of the present disclosure can be best understood when read in conjunction with the following drawings in which:

FIG. 1 is a schematic illustration of one or more embodiments of the integrated naphtha reforming and benzyl toluene production process of the present disclosure;

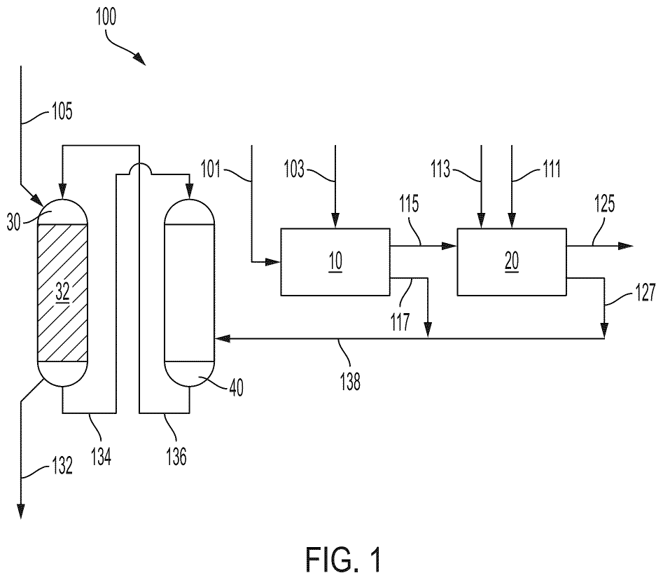

FIG. 2 is a schematic illustration of benzyl chloride generation according to one or more embodiments of the present disclosure;

FIG. 3 is a schematic illustration of benzyl toluene generation according to one or more embodiments of the present disclosure;

FIG. 4 is a schematic illustration of a catalyst regenerator according to one or more embodiments of the present disclosure;

FIG. 5 is a schematic illustration of one or more embodiments of the integrated naphtha reforming and benzyl toluene production process of the present disclosure including parallel naphtha reforming units;

FIG. 6 is a schematic illustration of one or more embodiments of the integrated naphtha reforming and benzyl toluene production process of the present disclosure including an aromatics complex;

FIG. 7 is a schematic illustration of one or more embodiments of the integrated naphtha reforming and benzyl toluene production process as illustrated in FIG. 6 including toluene recycling;

FIG. 8 is a schematic illustration of one or more embodiments of the integrated naphtha reforming and benzyl toluene production process as illustrated in FIG. 6 including reverse-trans alkylation of benzene and mixed xylenes to generate toluene; and

FIG. 9 is a schematic illustration of one or more embodiments of the integrated naphtha reforming and benzyl toluene production process as illustrated in FIG. 6 including hydrogenation of benzyl toluene to generate perhydro benzyl toluene.

For the purpose of these simplified schematic illustrations and the present description, the numerous valves, temperature sensors, electronic controllers and the like that are customarily employed and well known to those of ordinary skill in the art of certain refinery operations are not included. Further, accompanying components that are in conventional refinery operations such as, for example, air supplies, nitrogen supplies, hydrogen supplies, catalyst hoppers, and flue gas handling are not necessarily shown.

It should further be noted that arrows in the drawings refer to pipes, conduits, channels, or other physical transfer lines that connect by fluidic communication one or more system apparatuses to one or more other system apparatuses. Additionally, arrows that connect to system apparatuses define inlets and outlets in each given system apparatus.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

Reference will now be made in detail to embodiments of an integrated naphtha reforming and benzyl toluene production process and associated system of the present disclosure. While the integrated system for naphtha reforming and benzyl toluene production of the included figures are provided as exemplary, it should be understood that the present systems and methods may encompass other configurations.

The processes and systems of the present disclosure provide an integrated naphtha reforming and benzyl toluene production process and system. Specifically, the processes and systems of the present disclosure leverage the HCl generated as byproducts in production of benzyl toluene for the regeneration of reforming catalyst utilized in naphtha reforming. The synergy generated from the integration of the distinct hydrocarbon processing operations reduces costs, waste, and pollution for each individual process by converting a product waste stream to a value feedstock.

In one or more embodiments, an integrated naphtha reforming and benzyl toluene production process includes providing a first toluene stream 101 and a chlorine gas stream 103 to a halogenation reactor 10 and operating the halogenation reactor 10 to generate a benzyl chloride stream 115 and a first HCl effluent stream 117. The process further comprises providing the benzyl chloride stream 115, a second toluene stream 111, and a Lewis acid stream 113 to an alkylation reactor 20 and operating the alkylation reactor 20 to generate a benzyl toluene stream 125 and a second HCl effluent stream 127 through a Friedel-Crafts reaction. The process additionally comprises providing a naphtha stream 105 to a catalytic reforming unit 30, wherein a reforming catalyst 32 is disposed within the catalytic reforming unit 30. Additionally, the process includes operating the catalytic reforming unit 30 to generate a reformate stream 132 and a spent catalyst stream 134, the spent catalyst stream 134 representing the reforming catalyst 32 which has been used. The process further comprises providing the spent catalyst stream 134 and at least one of the first HCl effluent stream 117 and the second HCl effluent stream 127 to a catalyst regenerator 40 operating the catalyst regenerator 40 to regenerate the reforming catalyst 32 and form a regenerated reforming catalyst stream 136. Finally, the process comprises providing the regenerated reforming catalyst stream 136 as a recycle stream to the catalytic reforming unit 30 to replenish the reforming catalyst 32.

In one or more embodiments, an integrated system 100 for naphtha reforming and benzyl toluene production includes a catalytic reforming unit 30 comprising an input to receive a naphtha feed stream 105, a reforming catalyst 32 disposed within the catalytic reforming unit 30, and two or more outlets to discharge a spent catalyst stream 134 and a reformate stream 132, wherein the catalytic reforming unit 30 is configured to reform the naphtha feed stream 105 to generate the reformate stream 132 and the spent catalyst stream 134. The system 100 further comprises a catalyst regenerator 40 fluidly connected to the catalytic reforming unit 30 to receive the spend catalyst stream 134, wherein the catalyst regenerator 40 is configured to regenerate the spent catalyst in the spent catalyst stream 132. The system 100 also includes a halogenation reactor 10 comprising an input for receiving a first toluene stream 101, an input for receiving a chlorine stream 103, an output for discharging a first HCl effluent stream 117, and an output for discharging a benzyl chloride stream 117. The system 100 also includes an alkylation reactor 20 comprising an input to receive the benzyl chloride stream 115 from the halogenation reactor 10, an input to receive a Lewis acid stream 113, an input to receive a second toluene stream 111, an output to discharge a benzyl toluene stream 125, and an output to discharge a second HCl effluent stream 127, wherein the alkylation reactor 20 operates based on a Friedel-Crafts reaction. Finally, the system 100 includes one or more HCl recycling lines 138 fluidly connecting the catalyst regenerator 40 with the halogenation reactor 10 and the alkylation reactor 20 for transport of the first HCL effluent stream 117 and the second HCl effluent stream 127 to an input of the catalyst regenerator 40.

Having disclosed the basic operation of the integrated naphtha reforming and benzyl toluene production process and associated system, each step and unit operation of the embodiments of the integrated process and associated system are now provided in further detail.

### Benzyl Toluene Generation

Benzyl toluene production is generally achieved through a two-step process. In the first step, benzyl chloride is synthesized by reacting toluene with chlorine. Then, in a second step, benzyl chloride reacts with toluene through Friedel-Crafts alkylation to form benzyl toluene. Such process and associated unit operations are discussed in the present disclosure with separate discussion of the benzyl chloride generation and the benzyl toluene generation.

Benzyl chloride may be generated in accordance with embodiments of the present disclosure using any method known to those skilled in the art. Benzyl chloride is typically manufactured in industrial scale using thermal or photochemical chlorination of toluene. Specifically, irradiation using ultraviolet light or beta-radiation and the use of an elevated temperature such as 65-100° C. drives the reaction between toluene and chlorine gas to generate benzyl chloride. Such irradiation results in the excitation of chlorine molecules allowing them to split into two chloride ions and become available for reaction with the toluene. Specifically, a hydrogen is stripped from the methyl group of the toluene and replaced with a chloride ion. Further, hydrogen ion removed from the methyl group of the toluene may react with the remaining chloride ion to form HCl. The overall reaction is illustrated in Reaction 1, provide infra. Such reaction is carried out in the halogenation reactor 10.

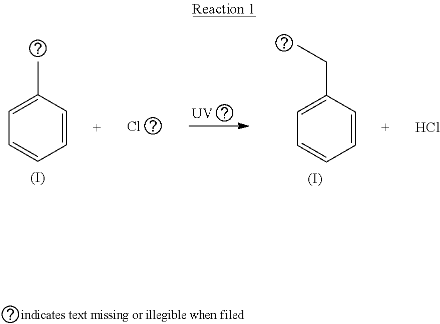

Benzyl toluene may then be generated from the benzyl chloride in accordance with embodiments of the present disclosure using any method known to those skilled in the art. Production of benzyl toluene from benzyl chloride is typically carried out through Friedel-Crafts reaction. Specifically, benzyl chloride reacts with toluene in the presence of a Lewis Acid. The overall reaction is illustrated in Reaction 2, provide infra. Such reaction is carried out in the alkylation reactor 20.

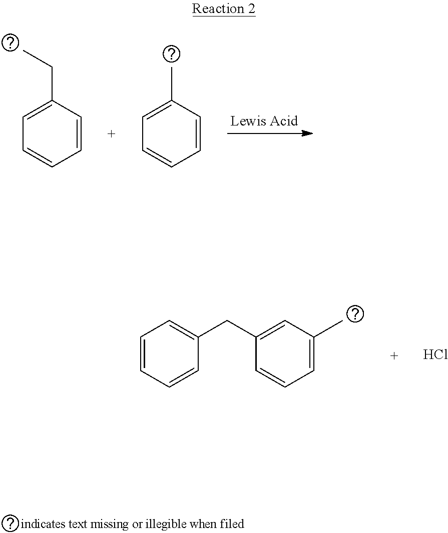

### Halogenation Reactor

Benzyl chloride is generated from toluene and chlorine gas in the halogenation reactor 10. In one or more embodiments, the halogenation reactor 10 comprises an input for receiving the first toluene stream 101, an input for receiving the chlorine stream 103, an output for discharging the first HCl effluent stream 117, and an output for discharging the benzyl chloride stream 115. It will be appreciated that in one or more embodiments the first HCl effluent stream 117 and the benzyl chloride stream 115 may be discharged from the halogenation reactor 10 as a single stream which is subsequently separated through any conventional means known to those skilled in the art to generate the separate first HCl effluent stream 117 and benzyl chloride stream 115. For conciseness such separation is not illustrated in the Figures provided with the present disclosure, but is within the understanding of one skilled in the art.

In one or more embodiments, the halogenation reactor 10 utilizes thermal chlorination of toluene to generate the benzyl chloride. For example, the halogenation reactor 10 may operate at 65° C. to 200° C., 100° C. to 200° C., or 65° C. to 100° C. to excite the chlorine within the chlorine stream 103 to generate chloride ions for reaction with the toluene provided in the first toluene stream 101.

In one or more embodiments, the halogenation reactor 10 utilizes photochemical chlorination of toluene to generate the benzyl chloride. For example, the halogenation reactor 10 may include a lamp which emits radiation in the ultraviolet range to excite the chlorine within the chlorine stream 103 to generate chloride ions for reaction with the toluene provided in the first toluene stream 101. In various embodiments, light may be provided from a bank of light emitting diodes (LEDs), a mercury vapor lamp, or other radiation sources. For examples the LEDs may emit radiation with wavelengths of 395 nm or 365 nm within the ultraviolet range. Similarly, the mercury vapor lamp may emit light in the wavelength range of 300 to 500 nm which corresponds to the region in which the absorption bands of chlorine lie.

In one or more embodiments, the halogenation reactor 10 is operated at substantially atmospheric pressure. Operation at atmospheric pressure and temperatures within the range discussed supra maintains toluene as a liquid and chlorine in a gaseous phase. However, at other temperature within the range discussed supra toluene and chlorine are both in a gaseous phase while benzyl chloride is in a liquid phase. Specifically, benzyl chloride has a boiling point of 179° C. and toluene has a boiling point of 110.6° C. generating an operating range between the boiling points where the benzyl chloride is a liquid and toluene is a gas. Under these conditions, benzyl chloride may be removed from the halogenation reactor 10 as it forms and condenses out.

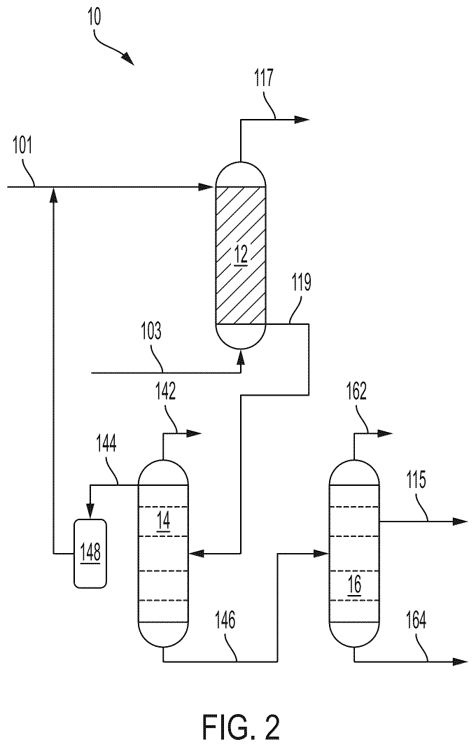

In one or more embodiments and with reference to FIG. 2, the halogenation reactor 10 comprises a chlorination unit 12 and a toluene recycling unit 14. The chlorination unit 12 converts the first toluene stream 101 to chlorinated compounds in a chlorination unit effluent 119 as well as HCl in the first HCl effluent stream 117. The toluene recycling unit 14 receives the chlorination unit effluent 119 from the chlorination unit 12 and separates unreacted toluene 144 from the chlorinated compounds in the chlorination unit effluent 119. The unreacted toluene 144 is recycled as a feed to the chlorination unit 12 and the chlorinated compounds are discharged for utilization or further processing.

It will be appreciated that the process of reacting chlorine and toluene to form benzyl chloride may result in the benzyl chloride being further chlorinated to form higher chlorinated compounds such as benzoyl chloride or benzotrichloride. To minimize formation of such higher chlorinated compounds toluene may be provided to the chlorination unit 12 in excess relative to the chlorine provided in the chlorine gas stream 103. Specifically, an excess of toluene allows most of the chlorine to be consumed to form benzyl chloride minimizing or eliminating formation of the higher chlorinated compounds. Specifically, benzyl chloride forms first before reacting with additional Cl₂ to form benzoyl chloride. It is a cascade reaction so consuming more chlorine at the first step restricts the second reaction to form benzoyl chloride. Accordingly, in one or more embodiments, the conversation to chlorinated compounds of the toluene stream including both the unreacted toluene 144 and the first toluene stream 103 fed to the chlorination unit 12 may be limited to 20 to 40% of the toluene input. However, according to various embodiments, the collection and recycling of the unreacted toluene 144 allows for conversion of the first toluene stream 103 initially provide to the chlorination unit 12 to reach 50%, 60%, 70%, 80%, 90%, 50-95%, 60-95%, 70-95%, 80-95%, or 85-95%.

In one or more embodiments, the chlorination reactor 12 may be operated at the same temperatures and pressures as detailed for the halogenation reactor 10 generally supra. For example, the chlorination reactor 12 may be operated at a temperature of 65 to 100° C. and atmospheric pressure.

The toluene recycling unit 14 receives the chlorination unit effluent 119 from the chlorination unit 12 and separates unreacted toluene 144 from the chlorinated compounds in the chlorination unit effluent 119. The chlorinated compounds which boil at a greater temperature than toluene are discharged as a chlorinated compound stream 146. Further, compounds boiling at a lower temperature than the toluene may be discharged from the toluene recycling unit 14 as a toluene separation off-gas 142. The toluene separation off-gas 142 comprises mostly HCl and accordingly, there is no need to purify this stream as small amounts of hydrocarbon or other chloride species will be burned when entering the catalyst regenerator 40. As such, the toluene recycling unit 14 generates streams of the toluene separation off-gas 142, the stream of unreacted toluene 144, and the chlorinated compound stream 146.

The toluene recycling unit 14 may comprise any unit operation or system known to those skilled in the art for separating a hydrocarbon stream by vapor pressure. An example toluene recycling unit 14 is an atmospheric distillation unit. An atmospheric distillation unit utilizes fractional distillation by heating the feed to a temperature at which one or more fractions of the mixture will vaporize while leaving other fractions as liquid to separate the feed stream. Further, in various embodiments, the toluene recycling unit 14 may be a simple flash column or true boiling point distillation with at least 15 theoretical plates.

In one or more embodiments, the toluene recycling unit 14 comprises a plurality of separation units. For ease of illustration, the provided FIG. 2 illustrate a single unit operation, but it will be appreciated that such unit operation may comprise multiple individual separator units to generate the disclosed product streams.

In one or more embodiments, the unreacted toluene 144 is provided to a toluene storage tank 148 to store the unreacted toluene 144 before transfer to the chlorination unit 12. The toluene storage tank 148 provides a buffer to account for variation in the rate of unreacted toluene 144 recovery from the chlorination unit effluent 119 while allowing for a steady-state feed to the chlorination unit 12.

In one or more embodiments, the chlorinated compounds are discharged from the toluene recycling unit 14 as the chlorinated compound stream 146. The chlorinated compound stream 146 may be provided to a benzyl chloride separation unit 16 for separation of benzyl chloride to form the benzyl chloride stream 115. Specifically, the benzyl chloride separation unit 16 may separate the benzyl chloride stream 115 from a lighter fraction of benzyl chloride separation off-gases 162 and a higher chlorinated fraction 164. The benzyl chloride separation off-gases 162 include the fractions boiling at less than benzyl chloride and the higher chlorinated fraction 164 includes the species boiling at a temperature greater than that for benzyl chloride. The higher chlorinated fraction may include benzoyl chloride and benzotrichloride representing toluene with two and three chlorinations respectively.

The benzyl chloride separation unit 16 may comprise any unit operation or system known to those skilled in the art for separating a hydrocarbon stream by vapor pressure. An example benzyl chloride separation unit 16 is an atmospheric distillation unit. Further, in various embodiments, the benzyl chloride separation unit 16 may be a simple flash column or true boiling point distillation with at least 15 theoretical plates. Further, in one or more embodiments, the benzyl chloride separation unit 16 comprises a plurality of separation units. For ease of illustration, the provided FIG. 2 illustrate a single unit operation, but it will be appreciated that such unit operation may comprise multiple individual separator units to generate the disclosed product streams.

### Alkylation Reactor

Benzyl toluene is generated from toluene and benzyl chloride along with a Lewis Acid in the alkylation reactor 20. In one or more embodiments, the alkylation reactor 20 comprises an input to receive the benzyl chloride stream 115 from the halogenation reactor 10, an input to receive the Lewis acid stream 113, an input to receive the second toluene stream 111, an output to discharge the benzyl toluene stream 125, and an output to discharge the second HCl effluent stream 127. Further, the alkylation reactor 20 operates based on a Friedel-Crafts reaction to generate the benzyl toluene stream 125. One skilled in the art is familiar with the mechanism of Friedel-Crafts reactions, and as such a detailed description of the mechanism is omitted. It will be appreciated that in one or more embodiments the second HCl effluent stream 127 and the benzyl toluene stream 125 may be discharged from the alkylation reactor 20 as a single stream which is subsequently separated through any conventional means known to those skilled in the art to generate the separate second the HCl effluent stream 127 and the benzyl toluene stream 125. For conciseness such separation is not illustrated in the Figures provided with the present disclosure, but is within the understanding of one skilled in the art.

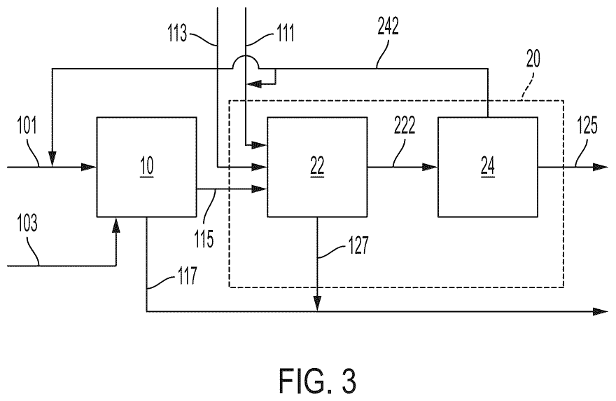

In one or more embodiments and with reference to FIG. 3, the alkylation reactor 20 comprises a benzylation unit 22 and a toluene recovery unit 24. The benzylation unit 22 converts the benzyl chloride stream 115 to benzyl toluene in a benzylation unit effluent 222 as well as HCl in the second HCl effluent stream 127. The toluene recovery unit 24 receives the benzylation unit effluent 222 from the benzylation unit 22 and separates residual toluene 242 from the benzyl toluene in the benzylation unit effluent 222. The residual toluene 242 may be recycled as a feed to the halogenation reactor 10 or the benzylation unit 22 and the benzyl toluene is discharged as the benzyl toluene stream 125 for utilization or further processing.

In one or more embodiments, the benzylation unit 22 completing the Friedel-Crafts reaction of the benzyl chloride stream 115 to generate benzyl toluene in the benzylation unit effluent 222 and HCl in the second HCl effluent stream 127 is operated at a temperature ranging from 50° C. to 150° C. In various further embodiments, the benzylation unit 22 may be operated at 70° C. to 150° C., 90° C. to 150° C., 120° C. to 140° C., or approximately 130° C.

The Friedel-Crafts reaction of the benzyl chloride stream 115 to generate benzyl toluene in the benzylation unit effluent 222 and HCl in the second HCl effluent stream 127 is completed in conjunction with the provision of the Lewis acid stream 113. The Lewis acid serves as a catalyst to drive the Friedel-Crafts reaction. In one or more embodiments, the Lewis acid stream 113 comprises one or more of ZnCl₂, FeCl₃, AlCl₃, SnCl₃, and TiCl₄. As such in one or more embodiments, the Lewis acid provided to the alkylation reactor 20, and more particularly the benzylation unit 22, may be selected from ZnCl₂, FeCl₃, AlCl₃, SnCl₃, and TiCl₄.

In one or more embodiments, the Lewis acid is provided to the benzylation unit 22 of the alkylation reactor 20 at 50 parts per million (ppm) to 50 percent by weight of the total feeds to the benzylation unit 22. In one or more further embodiments, a solid catalyst is packed in the alkylation reactor 20 negating the need for the Lewis acid stream 113.

To minimize formation of dibenzyltoluene and other higher order products, the total toluene provided to the benzylation unit 22 may be in excess relative to the benzyl chloride provided in the benzyl chloride stream 115 required for completion of the Friedel-Crafts reaction. Specifically, over alkylation can be a problem since the benzyl toluene is more reactive than the benzyl chloride and toluene. The alkylation may be controlled by provision of excess toluene to consume all chloride species.

The toluene recovery unit 24 may comprise any unit operation or system known to those skilled in the art for separating a hydrocarbon stream by vapor pressure. An example toluene recovery unit 24 is an atmospheric distillation unit. Further, in various embodiments, the toluene recovery unit 24 may be a simple flash column or true boiling point distillation with at least 15 theoretical plates. The residual toluene 242 removed by the toluene recovery unit 24 may be recycled as a feed to the halogenation reactor 10 or the benzylation unit 22 to reduce demand for fresh toluene to such unit operations.

In one or more embodiments, the benzylation unit effluent 222 consists essentially of benzyltoluene oligomers, an excess of toluene, and may contain chlorinated organic compounds such as chlorotoluenes, chlorobenzyltoluene and, in general, benzyltoluene oligomers bearing one or more chlorine atoms on the benzene nuclei. These compounds may have been introduced or are formed from impurities in the benzyl chloride or the Lewis acid. After removal of excess toluene in the toluene recovery unit 24, the generated benzyl toluene stream 125 may be further processed to remove the chlorinated organic compounds. For example, the benzyl toluene stream 125 may be treated with an alcoholate and heated with stirring to a temperature ranging from 220° C. to 320° C. After the dechlorination treatment, a single distillation may be performed to recover the benzyltoluene oligomers of low chlorine content. A heavy fraction containing the residues of the dechlorinating agent, NaCl, iron salts and heavy benzyltoluene oligomers remains as distillation bottoms.

### Catalytic Reforming Unit

Catalytic reforming is a chemical process used to convert petroleum refinery naphthas distilled from crude oil which typically have low octane ratings into high-octane liquid products termed reformates. The reformates are considered premium blending stocks for high-octane gasoline. Specifically, catalytic reforming converts low-octane linear hydrocarbons such as paraffins into branched alkanes such as isoparaffins and cyclic naphthenes, which are then partially dehydrogenated to produce high-octane aromatic hydrocarbons. As such, inclusion of catalytic reforming within hydrocarbon refineries is common.

In one or more embodiments, the catalytic reforming unit 30 comprises an input to receive a naphtha feed stream 105, a reforming catalyst 32 disposed within the catalytic reforming unit 30, and two or more outlets to discharge a spent catalyst stream 134 and a reformate stream 132. The catalytic reforming unit 30 is configured to reform the naphtha feed stream 105 to generate the reformate stream 132 and generates the spent catalyst stream 134 as a by-product of operation.

In one or more embodiments, the naphtha feed stream 105 is pre-treated to remove impurities, contaminants, and other species which may detrimentally affect operation of the catalytic reforming unit 30. Such pre-treatment may include a hydrotreatment or passage through filters. Such operations are within the scope and knowledge of one skilled in the art based on the particular characteristics of the naphtha feed stream 105.

The operation of the catalytic reforming unit 30 and the specific processing parameters of the naphtha feed stream 105 are outside the scope of the present disclosure. Specifically, the present disclosure is directed to integration of naphtha reforming generically and the associated process for regenerating spend catalyst generated as part of the naphtha reforming with benzyl toluene generation to achieve synergistic benefits. Accordingly, the catalytic reforming unit 30 may operate at a temperature, at a pressure, at a liquid hourly space velocity, or other operational parameter as desired or required for catalytic reforming of naphtha as understood by one skilled in the art.

The catalytic reforming unit 30 includes a reforming catalyst 32 disposed within the catalytic reforming unit 30. The catalytic bed reactor of the catalytic reforming unit 30 may operate may operate as a moving bed reactor in one or more embodiments. In further embodiments, the catalytic bed reactor of the catalytic reforming unit 30 may operate as a fixed bed reactor.

The catalytic reforming unit 30 may operate as a continuous regeneration reformer, as a semi-continuous regeneration reformer, or as a cyclic regeneration reformer. For purposes of this disclosure a continuous regeneration reformer operates in a continuous manner with spent catalyst continually removed and replaced with regenerated catalyst or fresh catalyst, a semi-continuous regeneration reformer operates continuously for a period before naphtha-reforming operations are discontinued and the catalyst is regenerated in situ, and a cyclic regeneration reformer operates with multiple reactors in parallel which may be cyclically taken off-line for regeneration while retaining the remaining reactors in operation.

As used herein, “spent catalyst” refers to catalyst which has undergone reaction with naphtha and is at least partially coked. Also, as used herein, “regenerated catalyst” refers to catalyst that is exiting the catalyst regenerator and is at least partially or substantially free of coke, and “fresh catalyst” refers to catalyst that is newly entering the system 100 and is at least partially or substantially free of coke.

Catalysts used in naphtha reforming processes are typically bifunctional and contain a metal for dehydrogenation and hydrogenation functionality and acid-catalyzed isomerization functionality. A metal site is provided by platinum and metals promoters such as rhenium, tin, germanium, and iridium. Further, an alumina support and chloride typically provide the acid-catalyzed isomerization functionality for the catalyst. In one or more embodiments, the reforming catalyst 32 is a platinum on alumina catalyst. In further embodiments, a zeolite and metal catalyst may be utilized with the zeolite provided as the acid site.

### Catalyst Regenerator

The catalyst regenerator 40 receives the spent catalyst stream 134 and regenerates the spent catalyst to generate the regenerated reforming catalyst stream 136. As previously indicated, the reforming catalyst 32 may be regenerated on a continuous or cyclical basis. The objective of catalyst regeneration is to restore the reforming catalyst 32 to a state similar to fresh catalyst where metal and acid sites are functioning as before coke deposition for use. Accordingly, catalyst regeneration typically includes burning off deposited coke in a controlled manner, redisposing platinum and promoter metals, and restoring catalyst chloride levels.

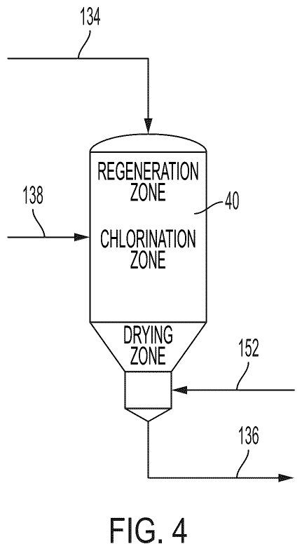

In one or more embodiments and with reference to FIG. 4, the catalyst regenerator 40 may operate with continuous regeneration of the reformer catalyst 32. The catalyst regenerator 40 with continuous regeneration may operation with three distinct zones: catalyst regeneration zone, a chlorination zone, and a drying zone. The spent catalyst stream 134 is provided to the top of the catalyst regenerator 40 and the spent catalyst passes sequentially through the zones and out the bottom of the catalyst regenerator as the regenerated reforming catalyst stream 136.

The catalyst regeneration zone removes the carbonaceous material and coke from the spent catalyst. Carbonaceous material or coke is deposited on the reforming catalyst 32 as an undesirable byproduct of the naphtha reforming process and the amount of carbonaceous deposit increases with time of operation, feed quality, and catalyst state. Accordingly, catalytic activity and selectivity performance also deteriorates as the platinum and acid sites become covered with coke. Thus, it is necessary to remove the coke during catalyst regeneration. In one or more embodiments, the spent catalyst is heated to a coke removal temperature of 500° C. to 600° C. in an environment with a controlled oxygen level to generate decoked catalyst. For example, in one or more embodiments, a nitrogen stream comprising 0.8 to 1.3 wt. % oxygen may be provided to provide the controlled oxygen environment to generate decoked catalyst. Regulation of the temperature and oxygen concentration permit controlled combustion of the catalyst coke during regeneration.

In one or more embodiments, the spent catalyst stream 134 may be passed through a disengaging hopper (not shown) to remove catalyst fines before provision to the catalyst regenerator 40. Further, the transfer of the spent catalyst to the catalyst regenerator 40 includes a hydrogen purge of the spent catalyst to remove entrained hydrocarbons and gases to allow for safe catalyst heating and regeneration in the catalyst regenerator 40.

Subsequent to decoking, the decoked catalyst is passed to a chlorination zone. The chlorination zone redisperses the platinum and replenishes chlorine to the alumina support. Even with controlled coke removal, the metal crystallites within the reforming catalyst 32 may become sintered during regeneration. As metal crystallites become larger in diameter, the activity of the reforming catalyst 32 decreases. As such, the redispersion of the metal, such as platinum, via an oxychlorination process is desired. Typically an organic chloride, such as dichloroethane, dichloropropane or tetrachloroethylene is used as a precursor to generate HCl and Cl instead of the direct provision of HCl in accordance with the present disclosure due to transportation and storage concerns. With dichloroethane as example, HCl is generated as an intermediate product to obtain chlorine through Deacon equilibrium. The chlorine then reacts with Pt oxide to form volatile species to achieve re-dispersion. However, direct provision of the HCl from the HCl recycling lines 138 fluidly connecting the catalyst regenerator 40 with the halogenation reactor 10 and the alkylation reactor 20 for transport of the first HCL effluent stream 117 and the second HCl effluent stream 127 allows for the initial steps of the oxychlorination to be skipped.

Redistribution of the platinum and replenishing chlorine to the alumina support in the reforming catalyst 32 is achieved according to Reaction 3 within the catalyst regeneration zone of the catalyst regenerator 40 followed by Reaction 4 and Reaction 5 within the chlorination zone of the catalyst regenerator 40. Specifically, platinum oxide is generated according to Reaction 3, Cl₂ is generated according to Reaction 4, and then the platinum oxide and the Cl₂ react to generate the regenerated reforming catalyst. Reactions 4 and 5 within the chlorination zone of the of the catalyst regenerator 40 to achieve oxychlorination may be conducted at an operating temperature of 450° C. to 550° C., 475° C. to 550° C., 450° C. to 525° C., 475° C. to 525° C., or approximately 500° C.

Ptₓ+xO₂↔(PtO₂)x Reaction 3

2HCl+½O₂↔Cl₂+H₂O(Deacon Equilibrium) Reaction 4

(PtO₂)x+Cl₂→(PtO₂)ₓ₋₁+Pt(O—Cl)₂ Reaction 5

In one or more embodiments, the reforming catalyst 32 passing from the chlorination zone of the catalyst regenerator 40 is further provided to a dying zone of the catalyst regenerator 40. The drying zone removes moisture adsorbed on the reforming catalyst 32 before recycling the reforming catalyst 32 back to the catalytic reforming unit 30. Water is a product of Reaction 4 and is desirably removed. The drying is achieved with flowing nitrogen provided from a nitrogen feed 152. It will be appreciated that the reforming catalyst 32 moving down from the chlorination zone of the of the catalyst regenerator 40 has temperature 450° C. to 550° C., so the temperature of the drying zone changes with a higher temperature at the entry of the drying zone and cooler at the exit of the drying zone.

It will be appreciated that omitting the generation of HCl from an organic chloride, such as dichloroethane, dichloropropane or tetrachloroethylene reduces carbon dioxide emissions from the process as such conversion generates carbon dioxide as a waste product. Further, providing the HCl from the benzyl toluene production eliminates the need to purchase HCl which directly reduces cost. Further, the direct consumption of the waste streams from the benzyl toluene production negates the need for transportation or storage when continuous regeneration is utilized.

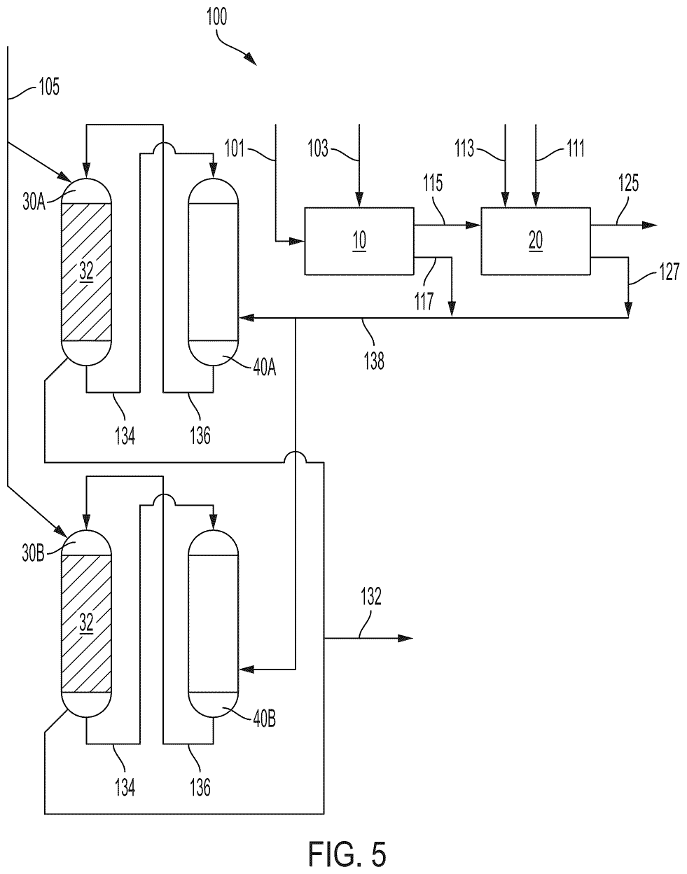

The naphtha reforming may also be achieved with utilization of multiple reactors. Accordingly, in one or more embodiments and with reference to FIG. 5, at least two catalytic reforming units 30 and at least two catalyst regenerators 40 may be provided in parallel to allow for cyclical operation of each processing train. Specifically, such arrangement allows for alternating operation of a first catalytic reforming unit 30A while regenerating the reforming catalyst 32 of a second catalytic reforming unit 40B and operation of the second catalytic reforming unit 30B while regenerating the reforming catalyst 32 of the first catalytic reforming unit 40A.

### Aromatics Complex

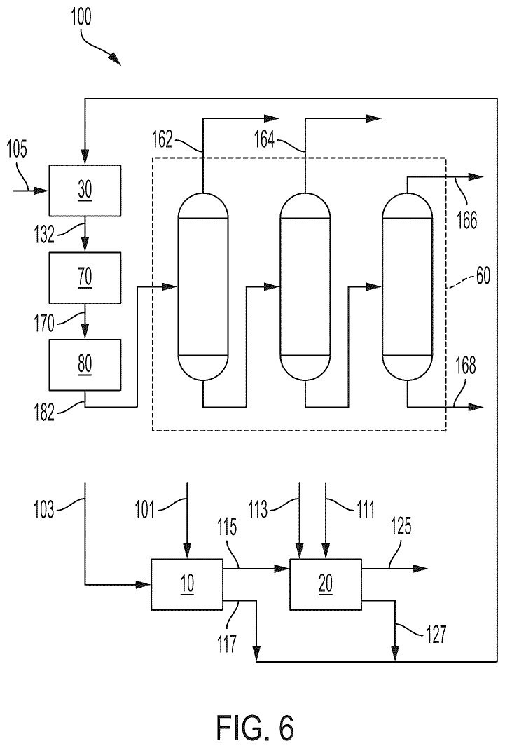

In one or more embodiments and with reference to FIGS. 6 through 9, the system 100 may include an aromatics complex 60 configured to receive and separate the reformate stream 132 into a benzene stream 162, a third toluene stream 164, and mixed xylenes stream 166.

Various systems and techniques may be utilized in the aromatics complex 60 for separating the reformate stream 132 into various fractions and the present disclosure is not intended to be limited in nature to the specific arrangement of the aromatics complex 60. Generally, the aromatics complex 60 produces the benzene stream 162, the third toluene stream 164, the mixed xylenes stream 166, as well as an aromatic bottoms fraction 168.

In some embodiments, benzene stream 162 may comprise benzene, a cyclic, aromatic hydrocarbon of formula C₆H₆. In embodiments, benzene stream 162 may comprise at least 80 wt. %, at least 90 wt. %, at least 95 wt. %, or at least 99 wt. % of benzene, on the basis of the total weight of benzene stream 162. In some embodiments, third toluene stream 164 may comprise toluene, a substituted cyclic, aromatic hydrocarbon of formula C₆H₅CH₃. In embodiments, the third toluene stream 164 may comprise at least 80 wt. %, at least 90 wt. %, at least 95 wt. %, or at least 99 wt. % of toluene, on the basis of the total weight of the third toluene stream 164. In some embodiments, mixed xylenes stream 166 may comprise mixtures of xylenes. Xylenes are a group of substituted cyclic, aromatic hydrocarbon of formula (CH₃)₂C₆H₄. In embodiments, mixed xylenes stream 166 may comprise at least 80 wt. %, at least 90 wt. %, at least 95 wt. %, or at least 99 wt. % of xylenes, on the basis of the total weight of mixed xylenes stream 166.

In one or more embodiments, the reformate stream 132 is passed through an aromatics extraction unit 70 to separate the reformate stream 132 into a non-aromatics fraction 172 and an aromatics fraction 170. As such, the aromatics fraction 170 may be passed to the aromatics complex 60 in lieu of the entire reformate stream 132. While the non-aromatics fraction 172 is illustrated as a single stream for reduced complexity in FIGS. 6 through 9, it will be appreciated that the non-aromatics fraction 172 may be further separated into individual steams of various constituent components. Similarly, while the aromatic bottoms fraction 168 is illustrated as a single stream for reduced complexity in FIGS. 6 through 9, it will be appreciated that the aromatic bottoms fraction 168 may be further separated into individual steams of various constituent components.

In one or more embodiments, the aromatics fraction 170 may be provided to a clay treatment unit 80 to remove unsaturated hydrocarbons from the aromatics fraction 170. The clay treatment unit 80 may operate to purify the aromatics fraction 170. In one or more embodiments, the clay treatment unit 80 may function to remove at least non-aromatic olefin compounds from the aromatics fraction 170 by reacting the non-aromatic olefin compounds by acid catalyzed alkylation. Generally, these non-aromatic olefin compounds may poison downstream units (such as p-xylene extraction units) or may reduce the purity of the product aromatic streams (benzene stream 162, third toluene stream 164, and mixed xylenes stream 166). The clay treatment unit 80 may function by contacting the aromatics fraction 170 with a Lewis acid catalyst, such as an activated clay. The clay treatment unit 80 may contact the aromatics fraction 170 with the Lewis acid catalyst at temperatures of greater than 165° C., such as greater than 170° C., greater than 180° C., from 165° C. to 250° C., from 170° C. to 230° C., from 180° C. to 22° C., from 190° C. to 210° C., or any subset thereof. The resulting clay treated stream 182 may be provided to the aromatics complex 60 in lieu of the entire reformate stream 132 or the aromatics fraction 170.

In one or more embodiments, the clay treated stream 182 may comprise benzene, toluene, xylenes, and C9+ aromatic compounds. In embodiments, the clay treated stream 182 may have a lower concentration of benzene and higher concentrations of toluene and xylenes than the aromatics fraction 170. In embodiments, the clay treated stream 182 may comprise at least 60 wt. %, at least 70 wt. %, at least 80 wt. %, at least 90 wt. %, at least 95 wt. %, or at least 99 wt. % of the combined weight of benzene, toluene, xylenes, and C9+ aromatic compounds, on the basis of the total weight of clay treated stream 182. In one or more embodiments, the clay treated stream 182 may comprise less than 5 wt. %, such as less than 2.5 wt. %, less than 1 wt. %, less than 0.5 wt. %, less than 0.25 wt. %, less than 0.1 wt. %, less than 0.01 wt. %, or even less than 0.001 wt. % of olefinic non-aromatic hydrocarbons, on the basis of the total weight of the clay treated stream 182.

There are many configurations of aromatic recovery complexes in general. In one or more embodiments, the aromatics complex 60 may include, for example, a dehexanizer distillation column that removes lighter components and discharges a bottoms product stream. The bottoms product stream may be fed to a benzene distillation column that removes benzene overhead and discharges a bottoms stream having, for example, toluene, mixed xylenes, ethyl benzene, and C9+ aromatic compounds. In some instances, the overhead discharge may enter absorber and stripper columns to purify the benzene. The bottoms stream from the benzene distillation column may be processed in absorber and stripper columns to remove light components and further in distillation columns. The aforementioned absorber and stripper columns may involve solvent extraction.

This bottoms stream from the benzene distillation column may ultimately be processed in distillation columns to separate and recover toluene and various mixed xylenes. The distillation columns may include a toluene distillation column(s) and a xylene distillation column(s). A toluene distillation column may separate and discharge toluene overhead. The xylene distillation column may receive the bottoms discharge from the toluene distillation column, separate and discharge mixed xylenes overhead and discharge a heavy aromatics (C9+) bottoms stream, such as the aromatic bottoms fraction 168.

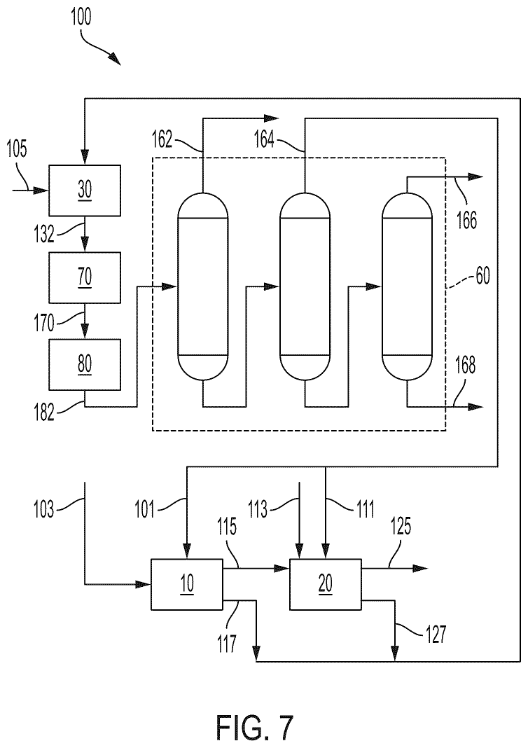

With integration of the aromatics complex 60, in one or more embodiments and with reference to FIG. 7, the toluene captured in the aromatics complex 60 may be provided to at least one of the halogenation reactor 10 and the alkylation reactor 20. Specifically, provision of the third toluene stream 164 to the halogenation reactor 10 and the alkylation reactor 20 at least partially offsets the demand for toluene from the first toluene stream 101 and the second toluene stream 111 respectively. Based on rate of toluene generation in the aromatics complex 60 and the demand for toluene for each of the halogenation reactor 10 and the alkylation reactor 20, in one or more embodiments, the third toluene stream 164 may provide make-up toluene to only one of the halogenation reactor 10 and the alkylation reactor 20 with an alternative toluene source supply the other. In is noted that in one or more embodiments, the halogenation reactor 10 and the alkylation reactor 20 may both be supplied from a single toluene feed line which includes valves and other standard piping equipment to direct a prescribed flow to each of the halogenation reactor 10 and the alkylation reactor 20. The third toluene stream 164 can be mixed with the single toluene feed line such that all sources of toluene to the halogenation reactor 10 and the alkylation reactor 20 are combined.

### Reverse Trans-Alkylation Reactor

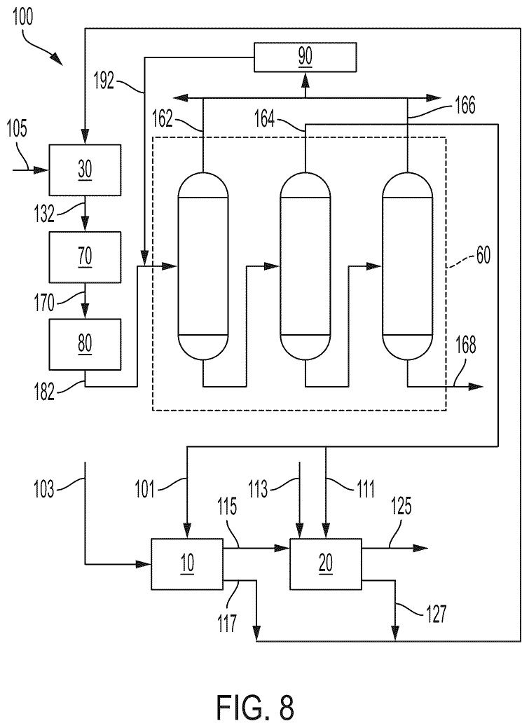

In one or more embodiments and with reference to FIG. 8, at least a portion of the benzene stream 162 and the mixed xylenes stream 166 captured in the aromatics complex 60 may be provided to a reverse trans-alkylation reactor 90 to convert the benzene and mixed xylenes to toluene. The toluene generated in the reverse trans-alkylation reactor 90 is discharged as a trans-alkylation reactor effluent stream 192.

In one or more embodiments, the trans-alkylation reactor effluent stream 192 is recycled as a feed to the aromatics complex 60. Specifically, the trans-alkylation reactor effluent stream 192 is rich in toluene and such toluene may be recovered within the aromatics complex 60. Generation of additional toluene with the trans-alkylation reactor 90 and subsequent recovery in the aromatics complex 60 allows for further integration of the naphtha reforming and benzyl toluene production processes and further reduces the demand for make-up toluene.

### Hydrogenation Unit

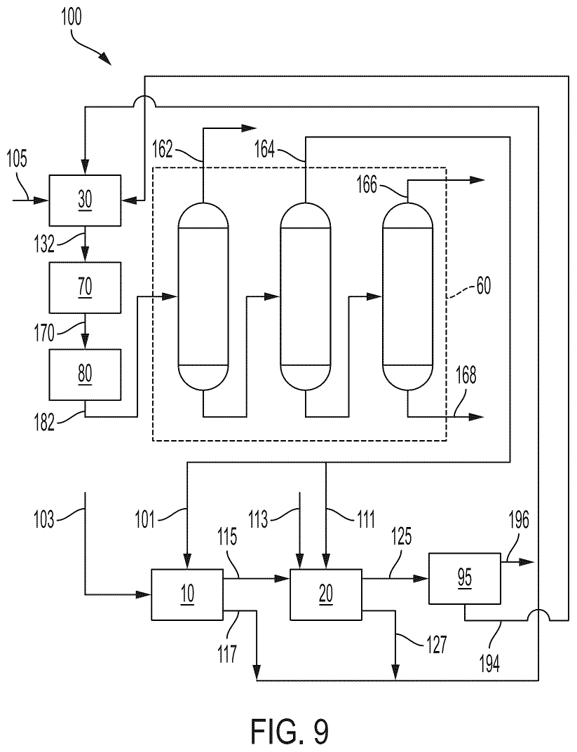

In one or more embodiments and with reference to FIG. 9, the benzyl toluene stream 125 generated in the alkylation reactor 20 may be provided to a hydrogenation unit 95. Passage of the benzyl toluene stream 125 to the hydrogenation unit 95 converts the benzyl toluene in the benzyl toluene stream 125 to perhydro benzyltoluene in a perhydro benzyltoluene stream 196.

In one or more embodiments, hydrogen generated in the catalytic reforming unit 30 may be provided to the hydrogenation unit 95. Specifically, a source of hydrogen is needed for operation of the hydrogenation unit 95 and utilization of a hydrogen stream 194 generated as a waste stream through normal operation of the catalytic reforming unit 30 further integrates the naphtha reforming and benzyl toluene production processes. Such processing reduces or eliminates demand for acquisition of hydrogen to operate the hydrogenation unit 95.

It should now be understood the various aspects of the integrated process and system for conversion of crude oil to value added petrochemicals are described and such aspects may be utilized in conjunction with various other aspects.

According to a first aspect, an integrated naphtha reforming and benzyl toluene production process includes (i) providing a first toluene stream and a chlorine gas stream to a halogenation reactor; (ii) operating the halogenation reactor to generate a benzyl chloride stream and a first HCl effluent stream; (iii) providing the benzyl chloride stream, a second toluene stream, and a Lewis acid stream to an alkylation reactor; (iv) operating the alkylation reactor to generate a benzyl toluenes stream and a second HCl effluent stream through a Friedel-Crafts reaction; (v) providing naphtha to a catalytic reforming unit, wherein a reforming catalyst is disposed within the catalytic reforming unit; (vi) operating the catalytic reforming unit to generate a reformate stream and a spent catalyst stream, the spent catalyst stream representing the used reforming catalyst; (vii) providing the spent catalyst stream and at least one of the first HCl effluent stream and the second HCl effluent stream to a catalyst regenerator; (viii) operating the catalyst regenerator to regenerate the reforming catalyst and form a regenerated reforming catalyst stream; and (ix) providing the regenerated reforming catalyst stream as a recycle stream to the catalytic reforming unit.

A second aspect includes the process of the first aspect in which both the first HCl effluent stream and the second HCl effluent stream are provided to the catalyst regenerator.

A third aspect includes the process of the first or second aspect in which the reformate stream is provided to an aromatics complex to separate and capture benzene, toluene, and mixed xylenes.

A fourth aspect includes the process of the third aspect in which the toluene captured in the aromatics complex is provided to at least one of the halogenation reactor and the alkylation reactor at least partially in lieu of the first toluene stream and the second toluene stream.

A fifth aspect includes the process of the third aspect in which the toluene captured in the aromatics complex is provided to both the halogenation reactor and the alkylation reactor at least partially in lieu of the first toluene stream and the second toluene stream.

A sixth aspect includes the process of any of the third through fifth aspects in which at least a portion of the benzene and the mixed xylenes captured in the aromatics complex is provided to a reverse trans-alkylation reactor to convert the benzene and mixed xylenes to toluene within a trans-alkylation reactor effluent stream.

A seventh aspect includes the process of the sixth aspect in which the trans-alkylation reactor effluent stream is recycled as a feed to the aromatics complex.

An eighth aspect includes the process of any of the first through seventh aspects in which the benzyl toluene generated in the alkylation reactor is provided to a hydrogenation unit, hydrogen generated in the catalytic reforming unit is provided to the hydrogenation unit, and the benzyl toluene is converted to perhydro benzyltoluene.

A ninth aspect includes the process of any of the first through eighth aspects in which the halogenation reactor generates benzyl chloride with thermal chlorination of toluene.

A tenth aspect includes the process of any of the first through ninth aspects in which the halogenation reactor generates benzyl chloride with photochemical chlorination of toluene.

An eleventh aspect includes the process of any of the first through tenth aspects in which the halogenation reactor comprises a chlorination unit and a toluene recycling unit, the chlorination unit converting toluene to chlorinated compounds and the toluene recycling unit separating unreacted toluene from the chlorinated compounds in an effluent of the chlorination unit; and the unreacted toluene is recycled as a feed to the chlorination unit achieving a benzyl chloride yield based on toluene fed to the halogenation reactor of at least 90%.

A twelfth aspect includes the process of any of the first through eleventh aspects in which the Lewis acid provided to the alkylation reactor is selected from ZnCl₂, FeCl₃, AlCl₃, SnCl₃, and TiCl₄.

A thirteenth aspect includes the process of any of the first through twelfth aspects in which excess toluene is provided to the alkylation reactor to consume chloride species and limit alkylation.

A fourteenth aspect includes the process of any of the first through thirteenth aspects in which the catalyst regenerator operates with continuous regeneration.

A fifteenth aspect includes the process of any of the first through thirteenth aspects in which at least two catalytic reforming units and at least two catalyst regenerators are provided in parallel, the process further comprising alternating operation of a first catalytic reforming unit while regenerating the reforming catalyst of a second catalytic reforming unit and operation of the second catalytic reforming unit while regenerating the reforming catalyst of the first catalytic reforming unit.

A sixteenth aspect includes the process of any of the first through fifteenth aspects in which in the catalyst regenerator the spent catalyst is heated to a coke removal temperature of 500° C. to 600° C. in a nitrogen stream comprising 0.8 to 1.3 wt. % oxygen to generate decoked catalyst.

A seventeenth aspect includes the process of the sixteenth aspect in which the reforming catalyst is a platinum on alumina catalyst.

An eighteenth aspect includes the process of the seventeenth aspect in which the decoked catalyst is passed to a chlorination zone the catalyst regenerator and combined with one or both of first HCl effluent stream and the second HCl effluent stream in the presence of oxygen at an operating temperature of 475° C. to 525° C. to redisperse platinum and replenish chlorine in the alumina support of the reforming catalyst.

A nineteenth aspect includes the process of any of the first through eighteenth aspects in which the reforming catalyst is further provided to a dying zone of the catalyst regenerator to remove moisture adsorbed on the catalyst before recycling the reforming catalyst back to the catalytic reforming unit.

According to a twentieth aspect, an integrated system for naphtha reforming and benzyl toluene production includes (i) a catalytic reforming unit comprising an input to receive a naphtha feed stream, a reforming catalyst disposed within the catalytic reforming unit, and two or more outlets to discharge a spent catalyst stream and a reformate stream, wherein the catalytic reforming unit is configured to reform the naphtha feed stream to generate the reformate stream and the spent catalyst stream; (ii) a catalyst regenerator fluidly connected to the catalytic reforming unit to receive the spend catalyst stream, wherein the catalyst regenerator is configured to regenerate the spent catalyst; (iii) a halogenation reactor comprising an input for receiving a first toluene stream, an input for receiving a chlorine stream, an output for discharging a first HCl effluent stream, and an output for discharging a benzyl chloride stream; (iv) an alkylation reactor comprising an input to receive the benzyl chloride stream from the halogenation reactor, an input to receive a Lewis acid stream, an input to receive a second toluene stream, an output to discharge a benzyl toluenes stream, and an output to discharge a second HCl effluent stream, wherein the alkylation reactor operates based on a Friedel-Crafts reaction; and (v) one or more HCl recycling lines fluidly connecting the catalyst regenerator with the halogenation reactor and the alkylation reactor for transport of the first HCL effluent stream and the second HCl effluent stream to an input of the catalyst regenerator.

A twenty-first aspect includes the system of the twentieth aspect in which the system further comprises an aromatics complex configured to receive and separate the reformate steam into a benzene stream, a third toluene stream, and mixed xylenes stream.

A twenty-second aspect includes the system of the twenty-first aspect in which the system further comprises a fluid connection between the halogenation reactor and the alkylation reactor for transfer of the third toluene stream from the aromatics complex to at least one of the halogenation reactor and the alkylation reactor at least partially in lieu of the first toluene stream and the second toluene stream.

It should be apparent to those skilled in the art that various modifications and variations can be made to the described embodiments without departing from the spirit and scope of the claimed subject matter. Thus, it is intended that the specification cover the modifications and variations of the various described embodiments provided such modifications and variations come within the scope of the appended claims and their equivalents.

For purposes of this disclosure, it is explicitly noted that indication that one stream or effluent is passed or provided from one unit to another unit includes embodiments where the stream or effluent is passed directly from one unit to another unit as well as embodiments where there is an intervening system or unit which may substantially change the composition of the stream or effluent between the units. As used in the present disclosure, passing a stream or effluent from one unit “directly” to another unit refers to passing the stream or effluent from the first unit to the second unit without passing the stream or effluent through an intervening reaction system or separation system that substantially changes the composition of the stream or effluent. Similarly, indication that two systems are “fluidly connected” indicates that streams may be passed directly between the systems. Heat transfer devices, such as heat exchangers, preheaters, coolers, condensers, or other heat transfer equipment, and pressure devices, such as pumps, pressure regulators, compressors, or other pressure devices, are not considered to be intervening systems that change the composition of a stream or effluent. Combining two streams or effluents together also is not considered to comprise an intervening system that changes the composition of one or both of the streams or effluents being combined.

It should further be understood that streams may be named for the components of the stream, and the component for which the stream is named may be the major component of the stream (such as comprising from 50 weight percent (wt. %), from 70 wt. %, from 90 wt. %, from 95 wt. %, from 99 wt. %, from 99.5 wt. %, or even from 99.9 wt. % of the contents of the stream to 100 wt. % of the contents of the stream). It should also be understood that components of a stream are disclosed as passing from one system component to another when a stream comprising that component is disclosed as passing from that system component to another. For example, a disclosed “hydrocarbon stream” passing to a first system component or from a first system component to a second system component should be understood to equivalently disclose “hydrocarbon” passing to the first system component or passing from a first system component to a second system component.

The singular forms “a”, “an” and “the” include plural referents, unless the context clearly dictates otherwise.

Throughout this disclosure ranges are provided. It is envisioned that each discrete value encompassed by the ranges are also included. Additionally, the ranges which may be formed by each discrete value encompassed by the explicitly disclosed ranges are equally envisioned. For brevity, the same is not explicitly indicated subsequent to each disclosed range and the present general indication is provided.

As used in this disclosure and in the appended claims, the words “comprise,” “has,” and “include” and all grammatical variations thereof are each intended to have an open, non-limiting meaning that does not exclude additional elements or steps.

1. An integrated naphtha reforming and benzyl toluene production process, the process comprising:
(i) providing a first toluene stream and a chlorine gas stream to a halogenation reactor;
(ii) operating the halogenation reactor to generate a benzyl chloride stream and a first HCl effluent stream;
(iii) providing the benzyl chloride stream, a second toluene stream, and a Lewis acid stream to an alkylation reactor;
(iv) operating the alkylation reactor to generate a benzyl toluene stream and a second HCl effluent stream through a Friedel-Crafts reaction;
(v) providing naphtha to a catalytic reforming unit, wherein a reforming catalyst is disposed within the catalytic reforming unit;
(vi) operating the catalytic reforming unit to generate a reformate stream and a spent catalyst stream, the spent catalyst stream representing the used reforming catalyst;
(vii) providing the spent catalyst stream and at least one of the first HCl effluent stream and the second HCl effluent stream to a catalyst regenerator;
(viii) operating the catalyst regenerator to regenerate the reforming catalyst and form a regenerated reforming catalyst stream; and
(ix) providing the regenerated reforming catalyst stream as a recycle stream to the catalytic reforming unit.

2. The process of claim 1, wherein both the first HCl effluent stream and the second HCl effluent stream are provided to the catalyst regenerator.

3. The process of claim 1, wherein the reformate stream is provided to an aromatics complex to separate and capture benzene, toluene, and mixed xylenes.

4. The process of claim 3, wherein the toluene captured in the aromatics complex is provided to at least one of the halogenation reactor and the alkylation reactor at least partially in lieu of the first toluene stream and the second toluene stream.

5. The process of claim 3, wherein the toluene captured in the aromatics complex is provided to both the halogenation reactor and the alkylation reactor at least partially in lieu of the first toluene stream and the second toluene stream.

6. The process of claim 3, wherein at least a portion of the benzene and the mixed xylenes captured in the aromatics complex is provided to a reverse trans-alkylation reactor to convert the benzene and mixed xylenes to toluene within a trans-alkylation reactor effluent stream.

7. The process of claim 6, wherein the trans-alkylation reactor effluent stream is recycled as a feed to the aromatics complex.

8. The process of claim 1, wherein:
the benzyl toluene generated in the alkylation reactor is provided to a hydrogenation unit;
hydrogen generated in the catalytic reforming unit is provided to the hydrogenation unit; and
the benzyl toluene is converted to perhydro benzyltoluene.

9. The process of claim 1, wherein the halogenation reactor generates benzyl chloride with thermal chlorination of toluene.

10. The process of claim 1, wherein the halogenation reactor generates benzyl chloride with photochemical chlorination of toluene.

11. The process of claim 1, wherein:
the halogenation reactor comprises a chlorination unit and a toluene recycling unit, the chlorination unit converting toluene to chlorinated compounds and the toluene recycling unit separating unreacted toluene from the chlorinated compounds in an effluent of the chlorination unit; and
the unreacted toluene is recycled as a feed to the chlorination unit.

12. The process of claim 1, wherein the Lewis acid provided to the alkylation reactor is selected from ZnCl₂, FeCl₃, AlCl₃, SnCl₃, and TiCl₄.

13. The process of claim 1, wherein excess toluene is provided to the alkylation reactor to consume chloride species and limit alkylation.

14. The process of claim 1, wherein the catalyst regenerator operates with continuous regeneration.

15. The process of claim 1, wherein at least two catalytic reforming units and at least two catalyst regenerators are provided in parallel, the process further comprising alternating operation of a first catalytic reforming unit while regenerating the reforming catalyst of a second catalytic reforming unit and operation of the second catalytic reforming unit while regenerating the reforming catalyst of the first catalytic reforming unit.

16. The process of claim 1, wherein in the catalyst regenerator the spent catalyst is heated to a coke removal temperature of 500° C. to 600° C. in a nitrogen stream comprising 0.8 to 1.3 wt. % oxygen to generate decoked catalyst.

17. The process of claim 16, wherein the reforming catalyst is a platinum on alumina catalyst.

18. The process of claim 17, wherein the decoked catalyst is passed to a chlorination zone the catalyst regenerator and combined with one or both of first HCl effluent stream and the second HCl effluent stream in the presence of oxygen at an operating temperature of 475° C. to 525° C. to redisperse platinum and replenish chlorine in the alumina support of the reforming catalyst.

19. The process of claim, 18, wherein the reforming catalyst is further provided to a dying zone of the catalyst regenerator to remove moisture adsorbed on the catalyst before recycling the reforming catalyst back to the catalytic reforming unit.

20. An integrated system for naphtha reforming and benzyl toluene production, the system comprising:
(i) a catalytic reforming unit comprising an input to receive a naphtha feed stream, a reforming catalyst disposed within the catalytic reforming unit, and two or more outlets to discharge a spent catalyst stream and a reformate stream, wherein the catalytic reforming unit is configured to reform the naphtha feed stream to generate the reformate stream and the spent catalyst stream;
(ii) a catalyst regenerator fluidly connected to the catalytic reforming unit to receive the spend catalyst stream, wherein the catalyst regenerator is configured to regenerate the spent catalyst;
(iii) a halogenation reactor comprising an input for receiving a first toluene stream, an input for receiving a chlorine stream, an output for discharging a first HCl effluent stream, and an output for discharging a benzyl chloride stream;
(iv) an alkylation reactor comprising an input to receive the benzyl chloride stream from the halogenation reactor, an input to receive a Lewis acid stream, an input to receive a second toluene stream, an output to discharge a benzyl toluene stream, and an output to discharge a second HCl effluent stream, wherein the alkylation reactor operates based on a Friedel-Crafts reaction; and
(v) one or more HCl recycling lines fluidly connecting the catalyst regenerator with the halogenation reactor and the alkylation reactor for transport of the first HCL effluent stream and the second HCl effluent stream to an input of the catalyst regenerator.

21. The system of claim 20, wherein the system further comprises an aromatics complex configured to receive and separate the reformate steam into a benzene stream, a third toluene stream, and mixed xylenes stream.

22. The system of claim 21, wherein the system further comprises a fluid connection between the halogenation reactor and the alkylation reactor for transfer of the third toluene stream from the aromatics complex to at least one of the halogenation reactor and the alkylation reactor at least partially in lieu of the first toluene stream and the second toluene stream.
